(19) United States
(12) Patent Application Publication
(10) Pub. No.: US 2009/0098201 A1
(43) Pub. Date: Apr. 16, 2009

(54) Composition and Method for Treatment and Prevention of Atherosclerosis

(75) Inventor: Howard J. Smith, Victoria (AU)

Correspondence Address: BIO INTELLECTUAL PROPERTY SERVICES (BIO IPS) LLC, 8509 KERNON CT., LORTON, VA, 22079, US

(73) Assignee: Bio Intellectual Property Services (Bio IPS) LLC, Lorton, VA (US)

(21) Appl. No.: 11/630,482

(22) PCT Filed: Jun. 27, 2005
(86) PCT No.: PCT/AU05/00938

§ 371 Date: Dec. 22, 2006

(30) Foreign Application Priority Data
Jun. 28, 2004 (AU) 2004903534

(51) Int. Cl. A61K 9/52; A61K 31/60; A61K 9/26; A61K 9/00; A61P 9/10

(52) U.S. Cl. 424/457; 514/165; 424/470; 424/486

(57) ABSTRACT

This invention relates to an oral composition for treatment or prevention of atherosclerosis comprising a low-dose aspirin and a low-dose of statin wherein the aspirin and statin are in a slow-release formulation. The invention also relates to a method of treatment or prevention of atherosclerosis using such a composition.

### FIELD

The present invention relates to a composition and method for treatment and prevention of atherosclerosis and its complications including coronary heart disease, cerebrovascular disease and peripheral vascular disease. In particular, the invention applies to the treatment of hypercholesterolaemia, the prevention of intravascular thrombosis, and the prevention of the interaction between hypercholesterolaemia and platelets.

### BACKGROUND

Atherosclerosis is the principal cause of arterial disease across the world, particularly the Western world, and it is the immediate cause of the modern epidemic of heart disease and stroke. Atherosclerosis is intimately related to both hypercholesterolaemia and to abnormalities of the intracellular handling of cholesterol by the intimal cells lining small arteries.

Key components of the pathology of atherosclerosis are:—
Elevation of the levels of cholesterol within the circulating blood.
Excessive entry of cholesterol into the intimal lining of small arteries.
Inadequate extrusion of cholesterol from arterial cells back into the plasma.
The intracellular response to cholesterol accumulation with the development of plaque, vascular thickening and fibrosis, and ulceration.
Progressive narrowing and eventual occlusion of small arteries causing reduction and cessation of blood flow.
Interaction between blood platelets and damaged arterial cells that initiates intravascular thrombosis and embolism with ischaemic damage to target organs.

In patients without any other cause of hypercholesterolaemia (such as liver or renal disease), elevation of plasma cholesterol has several components of which the main two are diet, and increased synthesis of cholesterol by the enzyme HMG-CoA reductase (3-hydroxy-3-methyl-glutaryl-coenzyme A reductase; HMG-CoA reductase). HMG-CoA reductase is the rate-limiting step in the conversion of 3-hydroxy-3-methyl-glutaryl Co A to mevalonate, from which cholesterol is then synthesised. The rate of synthesis of cholesterol is enhanced by genetically controlled activity of this enzyme, and by the consumption of saturated fats in the diet. Therefore, treatment with both dietary restriction of fats and weight control, supplemented by administration of HMG-CoA reductase inhibitors (statins) has become a key therapeutic strategy for both the prevention of atherosclerosis and the progression of established disease.

Statins such as simvastatin and lovastatin not only interfere with synthesis of cholesterol but also produce side effects which are a concern for long term prophylaxis and treatment. These side effects may include fatigue, myalgia, and less commonly limb weakness, myopathy and muscle necrosis.

Cholesterol does little or no harm until it penetrates intimal cells lining the arteries. Evidence suggests that excessive penetration of cholesterol follows damage to the cell membranes. Damage may be caused by several agents or processes including high blood pressure, smoking, oxidising agents, radiation and other toxic agents. A common feature of these processes is increase in the permeability of the cell membranes. Avoidance of these toxic agents and processes is part of the strategy of the treatment of the disease.

Like other cells, the intimal cells lining small arteries expend energy to control their internal milieu. As membranes become damaged, excessive calcium accumulation concentrates within mitochondria and acts to limit energy production and restrict extrusion of cholesterol. Various calcium antagonists, including nifedipine and diltiazem, have been shown to retard the progression of atherosclerosis presumably by inhibiting calcium entry into these cells.

The disease processes in which excessive cholesterol accumulation causes plaque, medial thickening, and ulceration are well known. These act together to cause progressive narrowing of the arteries with restriction of blood flow. Blood platelets are attracted to abnormal arterial cell membranes, particularly when these are ulcerated, and initiate first platelet aggregation and then thrombosis and embolism. Therefore, treatment with acetyl salicylic acid (aspirin) and other platelet active drugs, which reduce platelet stickiness, is now part of the management of vascular disease. The adherence of platelets to the arterial cell membrane also enhances the rate of phospholipid oxidation in the cell membranes. Therefore, platelet active agents that reduce platelet stickiness act to slow the rate of membrane damage as well as inhibiting the rate of intravascular thrombosis.

All treatments for prevention of atherosclerosis and its complications need to be administered in ways that will minimise the risk of unwanted side effects. In the case of statins, these include myopathy and muscle fatigue caused by depletion of ubiquinone (coenzyme Q10) in skeletal muscle.

Aspirin has been reported to have a role in reducing vascular disease however long term and administration of aspirin causes gastrointestinal problems of gastric erosion, ulceration, and gastrointestinal bleeding. These gastrointestinal problems can be reduced using enteric coatings so that aspirin does not have immediate contact in high concentration with the gastric or intestinal mucosa.

Aspirin decreases platelet adhesiveness by acylating cyclooxygenase of platelets but also inhibits endothelial cyclooxygenase thereby suppressing production of prostacyclin.

Brantmark et al studied the effect of different formulations of aspirin on platelet aggregation. They also examined effect of fast-pass clearance deacetylation. Following a study of bioavailability they concluded that rapid-release formulations of aspirin should be used in anti-platelet therapy because the drug is not usually detected in blood following intake of a slow-release formulation. They proposed that sufficient levels are required to provide overcome inactivation through deacetylation.

James et al also reported that lower dose of soluble aspirin rather than slow-release aspirin is necessary for optimal antithrombotic effect.

U.S. Pat. No. 6,235,311 describes a tablet comprising a statin cholesterol lowering agent and aspirin which are formulated together as a bilayer tablet to minimise interaction between the two actives. The statin and aspirin may have an enteric coating to avoid the problems of gastrointestinal bleeding associated with long-term aspirin therapy.

There is a need for a pharmaceutical composition that can be safely administered to subjects for long-term effective treatment and prevention of atherosclerosis while minimising the incidence of side effects.

### SUMMARY

In accordance with a first aspect, the invention provides a composition for treatment or prevention of atherosclerosis comprising a low-dose aspirin and at least one statin in low-dose wherein the aspirin and statin are in a slow-release formulation.

In accordance with a second aspect the invention provides a method for treatment or prevention of atherosclerosis in a subject comprising administering to the subject a composition comprising a low-dose aspirin and at least one statin in low-dose wherein the aspirin and statin are co-formulated as a slow-release formulation. The combined formulation is preferably administered to provide a daily dosage of aspirin of from 5 to 150 mg and preferably (in the case of simvastatin) from 20 to 100 mg and a daily dosage of statin the range of from 0.5 to 100 mg and preferably from 5 to 80 mg. The invention also applies to equivalent doses of other statins.

In a third aspect the invention provides the use of a low-dose aspirin and at least one statin in low-dose in preparation of a slow-release formulation for treatment or prevention of atherosclerosis.

In a preferred aspect the invention provides unit dose form such as a tablet capsule or the like comprising from 5 to 150 mg of aspirin (preferably 20 to 100 mg and most preferably 20 to 80 mg) and from 0.5 to 100 mg (more preferably 5 to 80 and most preferably 10 to 60 mg) of a statin (simvastatin or the equivalent dose of other statins) wherein the aspirin and statin are in slow-release formulation. The slow-release form will generally be control-release to provide a sustained level of each in the portal vein sufficient for aspirin and statin to exhibit a therapeutic effect in the portal and avoid significant levels in the systemic vasculature by virtue of hepatic clearance. In this way we have found that the action is selectively exerted in the liver and portal vein albeit that the therapeutic effects such as reduction in platelet stickiness and lowering of cholesterol together significantly reduce or prevent vascular disease.

In contrast to the characteristics of the slow-release formulation of other drugs, which usually release product over 4-8 hours, the preferred slow-release co-formulation for statin and aspirin will release product over longer periods of time, preferably more than 12 hours, so that the concentration of both drugs in the liver and portal vein is prolonged for a substantial part of the 24-hour day

### DETAILED DESCRIPTION

The co-formulation product provides both the statin (HMG-CoA reductase inhibitor) and the aspirin presented as a liver-selective formulation to achieve therapeutic concentrations within the liver but low concentrations in the rest of the body. Thus, the action of the HMG-CoA reductase inhibitor within the portal vein and inhibits the formation of cholesterol without inhibiting formation of ubiquinone (coenzyme Q10) in the rest of the body. At the same time, the aspirin acts on the blood platelets during their transit through the portal vein to inhibit formation of thromboxane A₂ within the platelets and thereby reduce platelet stickiness. However, the low concentrations of aspirin in the rest of the body are insufficient to inhibit synthesis of prostacyclin, which is a systemic vasodilator substance. Thus, administration of aspirin to the body within a liver-selective formulation effectively makes the effect of aspirin platelet-selective and reduces platelet stickiness without other effects. Because the drug is presented as a slow-release formulation, it is released as the composition in a suitable form, such as capsule or tablet, descends through the gastrointestinal tract thereby reducing contact between the bowel lumen and higher concentrations of aspirin. This acts to reduce the risk of stomach and bowel erosion. The dosage form may if desired also have an enteric coating to inhibit gastrointestinal damage.

The inhibition of the synthesis of cholesterol by statins and the inhibition of platelet stickiness by aspirin are complementary effects that act together to retard the progression of atherosclerosis and help prevent its complications.

The dose and release rate of each of aspirin and statin are preferably low enough so that there is virtually complete hepatic clearance of the drugs before entry of blood to the systemic vasculature. The drugs therefore exhibit a selective effect with no significant detrimental effects in the systemic vasculature.

In accordance with another aspect of the present invention, we provide a method of pharmaceutical therapy comprising the administration of a liver-selective co-formulation providing slow-release of a low-dose of a statin and slow-release of a low-dose of aspirin. Administered together in this way, the complementary actions of statins and aspirin act to prevent or retard the development of atherosclerosis and prevent or reduce the rate of thrombo-embolic complications, but at the same time have minimal side effects that are related respectively to inhibition by statins of ubiquinone synthesis in skeletal muscle and the systemic effects of aspirin on the gastrointestinal tract and the systemic vasculature.

The concept of liver-selective drug delivery requires that a drug with a short half-life be administered as low-dose and as a slow-release or controlled-release formulation so that the drug is released slowly over several hours—preferably up to 24 hours. After crossing the gastrointestinal wall, the drug reaches the relatively small volume of the portal venous system and is carried to the liver. Here a significant portion is removed from the circulation by metabolism with the remainder passing into the much larger volume systemic circulation. In this way, a stable concentration gradient is achieved where the concentration of the drug is up to 5 or more times higher in the liver and portal circulation than in the systemic circulation.

Presentation as a liver-selective formulation also uses drugs that are reliably absorbed across the gastrointestinal wall after release from a capsule or other formulation descending through the gastrointestinal tract. Lipophilic agents cross cell membranes readily thereby fulfilling these criteria.

The invention provides a formulation providing slow-release of a low dose of aspirin and slow-release of a low dose of statin.

The US National Formulatory has recognised clear distinction between timed-release tablets and capsules and “enteric coated”. For example N.F. XIII 882 (1970) described the dosage forms as follows:
“As understood herein, time-release would include those tablets and capsules variously known as ‘delayed action’, ‘extended-release’, ‘prolonged action’ or ‘repeat action’, but would not include tablets specifically identified as ‘enteric coated’.” [N.F. XIII, 8821970)].

As used herein the terms slow-release, controlled-release and sustained-release therefore do not include compositions merely containing an enteric coating.

In previous therapies of aspirin and/or statins it has generally been assumed that the effects of these drugs are intimately related to the dose of drug administered. I have found in accordance with the invention that duration of exposure to aspirin and/or statins in the relatively small volume hepatic portal vein is the key factor.

Without wishing to be bound by theory the period of exposure in the portal vein is believed to be the critical factor because production of platelets is continuous and contact with relatively high therapeutic concentrations of the drug in the portal vein is sufficient to provide a clinical effect despite the small and generally subclinical systemic levels of the drug.

Thus while enteric coatings of aspirin and/or statin are helpful in reducing gastric erosion and bleeding enteric coatings do not provide the prolonged slow-release necessary to take advantage of selectivity provided by the difference in volume of the portal and systemic vascular system and the high clearance of the drugs in the liver.

The therapeutically effective amounts and release rates of statin to be used in compositions and the method of the invention will depend on the specific statin, and the patient including the age and condition of the patient. The therapeutically effective amount will be an amount which in combination with the aspirin provides a therapeutic benefit in prevention or treatment of one or more cardiovascular disease. The amount may be an amount useful for lowering low density lipoprotein (LDL-C).

The dose and release rate of statin are generally low enough to allow virtually complete hepatic clearance. Consequently the levels of drug remaining in the systemic vasculature are less than required to provide a systemic effect.

The therapeutically effective amount and dose delivery rate of aspirin will provide a reduction in the adhesiveness of platelets. Aspirin acts to inhibit the enzyme cyclooxygenase, and thence to inhibit the synthesis of both thromboxane (TXA₂) and prostacyclin (PGI₂).

TXA₂, which is produced in platelets by cyclooxygenase, increases platelet aggregation and induces vasoconstriction. Aspirin acts to inhibit both of these processes. TXA₂ is metabolised to TXB₂, so that measurement of this metabolite and a reduction in its plasma level, can be used to monitor the inhibitory effects of aspirin on TXA₂ synthesis.

By contrast PGI₂ is produced in the endothelium of blood vessels, particularly arterioles, and contributes to vasodilation. Aspirin also acts to inhibit these processes. PGI₂ is metabolised to PGF_{1α}, so that measurement of this metabolite and a reduction in its plasma level, can be used to monitor the inhibitory effects of Aspirin on PGI₂ synthesis.

The plasma ratio of TXB₂ to PGF_{1α}, and more particularly, changes in the ratio, can therefore be used as a measure of the relative effects of Aspirin on platelets and blood vessels. That is, a fall in the TXB₂:PGF_{1α} ratio after administration of an antiplatelet medication indicates that a relatively selective inhibitory effect on platelets has been achieved. By contrast, if administration of aspirin causes a reduction in both TXB₂ and PGF_{1α}, that is, with no significant change in the TXB₂:PGF_{1α} ratio, a non-selective effect on both platelets and blood vessels has been achieved. It is preferred that the treatment of the invention bring about a three fold change to provide a highly selective inhibitory effect on platelets.

In the present invention the platelet loading process with aspirin is sustained but restricted to the portal circulation to achieve inhibition of synthesis of TXA₂ in platelets but with minimal inhibition of PGI₂ in the systemic blood vessels. Administered in this way, the formulation aspirin may be described as “platelet-selective” or “thromboxane-selective” or “prostacyclin-sparing”.

The statin used in the composition and method of the invention preferably has a relatively short half-life. Simvastatin and fluvastatin are examples of HMG-CoA reductase inhibitors with relatively short half-lives suitable for administration as a liver-selective formulation. The short half-life (3-4 hours) is the result of hepatic metabolism to inactive metabolites. The dose required will be about 25% of the usual systemic dose of these agents. Most preferably the daily dose of aspirin is in the range of from 5 to 50 mg and most preferably the daily dose of statin, for example in the case of simvastatin, is in the range of from 5 to 50 mg. The doses of statins may vary depending on the particular statin used however the invention uses doses significantly less than those used in current therapy that employs systemic doses. The concept of a liver-selective formulation of a HMG-CoA reductase inhibitor has been described by me in US Patent Application 20020160044.

Aspirin is well absorbed from the stomach and small bowel and is converted to salicylate (salicylic acid primarily in the mucosa of the small bowel. It is then metabolised within the liver to three main metabolic products. Previous studies suggest that a dose of 50 mg administered as a slow-release formulation will inhibit the formation of thromboxane without inhibition of prostacyclin. Although some have claimed that the action of aspirin is relatively selective for the platelet especially when given in the low-dose of 50 mg/day (Carlsson et al, 1990), the inhibition of prostacyclin, which operates within the vasculature, also remains significant (Kyrle et al, 1989). It is believed that both the degree and duration of the effect of aspirin on platelets are a function of duration of exposure to the platelets more than the total dose administered.

The administration of aspirin as a low-dose slow-release formulation in the form of a liver-selective formulation means that it is effectively a platelet-selective formulation and the present invention relates to the co-prescription or co-formulation of such an agent with a slow-release, or liver-selective formulation of an HMG-CoA reductase inhibitor (statin).

The invention preferably uses statins that are poorly water-soluble. Such statins include statin compounds in their lactone forms and derivatives thereof having a water solubility of less than 5 mg per litre of water.

Particularly preferred poorly water-soluble statins include simvastatin, lovastatin, derivatives thereof and their pharmaceutically acceptable salts.

The more preferred statins for use in the present invention are simvastatin and lovastatin. The appropriate dose of statin will depend on the drug and the condition of the patient such as the age and health. Typically the systemic bioavailability of statin is less than about 5% by weight and preferably the systemic bioavailability of aspirin from the formulation used should also be low.

The inhibition of the synthesis of cholesterol by statins and the inhibition of platelet stickiness by aspirin are complementary effects that act together to prevent and retard the progression of atherosclerosis and its complications. Moreover the present invention combines a liver-selective statin component and platelet-selective aspirin component together to provide a treatment suitable for long-term treatment of cardiovascular disease without the problems caused by side effects and cumulative side effects of the drug combination. The complementary nature of their therapeutic actions has at least four components.
1) The HMG-CoA reductase inhibitors (statins) act to lower plasma cholesterol and thereby retard the progression of atherosclerosis.
2) The aspirin acts to reduce platelet stickiness and thereby reduces the risk of platelet aggregation and thromboembolic complications.
3) The lowering of plasma cholesterol by the statins increases the sensitivity of the platelets to nitric oxide, and thence to aspirin (Stepien et al, 2003).
4) By reducing platelet stickiness and adhesion of platelets to the intimal lining of arteries, aspirin reduces oxidative damage to the phospholipid membranes of the arterial cells, and thereby reduces cholesterol entry into the cells.
Thus, aspirin complements the effect of the statins by reducing the entry of cholesterol into the cells, and statins complement the effects of aspirin by making the platelets more sensitive to nitric oxide and the action of aspirin.

### Formulation for Slow-Release

There are many techniques to effect slow-release of an active pharmaceutical agent from an orally-administered formulation. These methods may include techniques designed to delay the disintegration of a capsule, tablet, or other vehicle, techniques designed to delay the solubility of a capsule, tablet or other vehicle, and techniques in which an active agent may be bound to a polymer or other large molecule such that absorption can not take place until the substance has been released from the polymer or other large molecule. The means of achieving these different methods of slow-release are varied and include well-known older methods, such as layers of shellac coating, and more modern techniques using synthetic and cellulose polymers.

The dosage forms according to the present invention may be controlled-release dosage forms. The mechanism of release of these dosage forms can be controlled by diffusion and or erosion. In some embodiments, the formulation comprises polymer-coated multiparticulates, polymer-coated tablets or minitablets, or hydrophilic matrix tablets.

A slow-release formulation of a HMG-CoA reductase agent plus aspirin designed as a preventive and protective agent against atherosclerosis may be designed to release the drug over a period of about 6 to about 24 hours following administration, thereby permitting once-a-day administration and providing a sustained exposure of the drug to the liver and to the platelets. In some embodiments, formulations releasing the drug over extended periods of time may have more than one timed-release component to effect time coverage.

The composition of the invention provides slow-release of aspirin and statin. The terms sustained-release or extended-release are also used in the art to refer to slow-release formulations.

General methods of providing slow or extended-release of active agents are well known in the art and having regard to the nature of the actives agents a skilled person will have no difficulty in providing suitable compositions to meet the desired release characteristics. Generally the composition of the invention will provide sufficiently slow-release such that not more than 50% of each of aspirin and statin is released within two hours preferably not more than 50% is released within three hours.

There are a number of general strategies known in the art for slow-release formulation which may be adopted for use in the present invention. Slow-release formulations of statins and aspirin may be produced by combining separately prepared slow-release particles or granules of each or by incorporating each in a multiplayer or homogeneous composition.

Certain statins such as pravastatin are acid labile and it is preferred to formulate such statins separately from aspirin to provide an intermediate composition such as slow-release granules and to combine these intermediate compositions in a unit dose such as by compressing the mixture containing the appropriate proportion to form a tablet or using the appropriate proportion of granules to fill a capsule such as a gelatine or other suitable capsule body.

In one embodiment of the slow-release formulation the aspirin and statin are separately or together incorporated into a slow-release matrix. The slow-release matrix preferably comprises a mixture of water-soluble and water-insoluble polymers.

Suitable water-soluble polymers include, but are not limited to, polyvinyl alcohol, polyvinylpyrrolidone, methylcellulose, hydroxypropylcellulose, hydroxypropylmethyl cellulose or polyethylene glycol, and/or mixtures thereof.

Suitable water-insoluble polymers include, but are not limited to, ethylcellulose, cellulose acetate cellulose propionate, cellulose acetate propionate, cellulose acetate butyrate, cellulose acetate phthalate, cellulose triacetate, poly (methyl methacrylate), poly (ethyl methacrylate), poly (butyl methacrylate), poly (isobutyl methacrylate), and poly (hexyl methacrylate), poly (isodecyl methacrylate), poly (lauryl methacrylate), poly (phenyl methacrylate), poly (methyl acrylate), poly (isopropyl acrylate), poly (isobutyl acrylate) poly (octadecyl acrylate), poly (ethylene), poly (ethylene) low density, poly (ethylene) high density, poly (ethylene oxide), poly (ethylene terephthalate), poly (vinyl isobutyl ether), poly (vinyl acetate), poly (vinyl chloride) and polyurethane, and/or mixtures thereof. Waxes, paraffins and the like, are also included in this group.

Suitable pharmaceutically acceptable excipients include, but are not limited to, carriers, such as sodium citrate and dicalcium phosphate; fillers or extenders, such as stearates, silicas, gypsum, starches, lactose, sucrose, glucose, mannitol, talc, and silicic acid; binders, such as hydroxypropyl methylcellulose, hydroxymethyl-cellulose, alginates, gelatin, polyvinyl pyrrolidone, sucrose and acacia; humectants, such as glycerol; disintegrating agents, such as agar, calcium carbonate, potato and tapioca starch, alginic acid, certain silicates, EXPLOTAB™, crospovidone, and sodium carbonate; solution retarding agents, such as paraffin; absorption accelerators, such as quaternary ammonium compounds; wetting agents, such as sodium lauryl sulfate, cetyl alcohol, and glycerol monostearate; absorbents, such as kaolin and bentonite clay; lubricants, such as talc, calcium stearate, magnesium stearate, solid polyethylene glycols, and sodium lauryl sulfate; stabilizers, such as fumaric acid; coloring agents; buffering agents; dispersing agents; preservatives; organic acids; and organic bases. The aforementioned excipients are given as examples only and are not meant to include all possible choices. Additionally, many excipients may have more than one role, or be classified in mole than one group; the classifications are descriptive only, and not intended to limit any use of a particular excipient.

The amounts and types of polymers, and the ratio of water-soluble polymers to water-insoluble polymers in the formulations are generally selected to achieve a desired release profile of the at least one simvastatin and/or lovastatin, as described below. For example, by increasing the amount of water insoluble-polymer relative to the water soluble-polymer, the release of the drug may be delayed or slowed. This is due, in part, to an increased impermeability of the polymeric matrix, and, in some cases, to a decreased rate of erosion during transit through the GI tract.

It may be preferred to separately form slow-release particles of aspirin and statin and to compress them into a tablet or fill a capsule with granules. In this way the optimum release can be provided for each of the statin and aspirin to extend the release profile of each.

The modified release formulations of the present invention may also be provided as membrane-controlled formulations. Membrane-controlled formulations of the present invention can be made by preparing a rapid release core, which may be a monolithic (e.g., tablet) or multi-unit (e.g., pellet) type, and coating the core with a membrane. The membrane-controlled core can then be further coated with a functional coating. In between the membrane-controlled core and the functional coating, a barrier or sealant may be applied. With this as an overview, details of membrane-controlled dosage forms are provided below.

The modified release formulations of the present invention may comprise at least one polymeric material, which can be applied as a membrane coating to the drug-containing cores. Suitable water-soluble polymers include, but are not limited to, polyvinyl alcohol, polyvinylpyrrolidone, methylcellulose, hydroxypropylcellulose, hydroxypropylmethyl cellulose or polyethylene glycol, and/or mixtures thereof.

Suitable water-insoluble polymers include, but are not limited to, ethylcellulose, cellulose acetate cellulose propionate, cellulose acetate propionate, cellulose acetate butyrate, cellulose acetate phthalate, cellulose triacetate, poly (methyl methacrylate), poly (ethyl methacrylate), poly (butyl methacrylate), poly (isobutyl methacrylate), and poly (hexyl methacrylate), poly (isodecyl methacrylate) poly (lauryl methacrylate), poly (phenyl methacrylate), poly (methyl acrylate), poly (isopropyl acrylate), poly (isobutyl acrylate), poly (octadecyl acrylate), poly (ethylene), poly (ethylene) low density, poly (ethylene) high density, poly (ethylene oxide), poly (ethylene terephthalate), poly (vinyl isobutyl ether), poly (vinyl acetate), poly (vinyl chloride) or polyurethane, and/or mixtures thereof.

EUDRAGIT™ polymers (available from Rohm Pharma) are polymeric lacquer substances based on acrylates and/or methacrylates. A suitable polymer that is freely permeable to the active ingredient and water is EUDRAGIT™ RL. A suitable polymer that is slightly permeable to the active ingredient and water is EUDRAGIT™ RS. Other suitable polymers which are slightly permeable to the active ingredient and water, and exhibit a pH-dependent permeability include, but are not limited to, EUDRAGIT™ L, EUDRAGIT™ S, and EUDRAGIT™ E.

EUDRAGIT™ RL and RS are acrylic resins comprising copolymers of acrylic and methacrylic acid esters with a low content of quaternary ammonium groups. The ammonium groups are present as salts and give rise to the permeability of the lacquer films. EUDRAGIT™ RL and RS are freely permeable (RL) and slightly permeable (RS), respectively, independent of pH. The polymers swell in water and digestive juices, in a pH-independent manner. In the swollen state, they are permeable to water and to dissolved active compounds.

EUDRAGIT™ L is an anionic polymer synthesized from methacrylic acid and methacrylic acid methyl ester. It is insoluble in acids and pure water. It becomes soluble in neutral to weakly alkaline conditions. The permeability of EUDRAGIT™ L is pH dependent. Above pH 5.0, the polymer becomes increasingly permeable.

In one embodiment comprising a membrane-controlled dosage form, the polymeric material comprises methacrylic acid co-polymers, ammonio methacrylate co-polymers, or a mixture thereof. Methacrylic acid co-polymers such as EUDRAGIT™ S and EUDRAGIT™ L (Rohm Pharma) are suitable for use in the controlled release formulations of the present invention, these polymers are gastroresistant and enterosoluble polymers. Their polymer films are insoluble in pure water and diluted acids. They dissolve at higher pHs, depending on their content of carboxylic acid. EUDRAGIT™ S and EUDRAGIT™ L can be used as single components in the polymer coating or in combination in any ratio. By using a combination of the polymers, the polymeric material may exhibit a solubility at a pH between the pHs at which EUDRAGIT™ L and EUDRAGIT™ S are separately soluble.

The membrane coating may comprise a polymeric material comprising a major proportion (i.e., greater than 50% of the total polymeric content) of one or more pharmaceutically acceptable water-soluble polymers, and optionally a minor proportion (i.e., less than 50% of the total polymeric content) of one or more pharmaceutically acceptable water insoluble polymers. Alternatively, the membrane coating may comprise a polymeric material comprising a major proportion (i.e., greater than 50% of the total polymeric content) of one or more pharmaceutically acceptable water insoluble polymers) and optionally a minor proportion (i.e., less than 50% of the total polymeric content) of one or more pharmaceutically acceptable water-soluble polymers.

Ammonia methacrylate co-polymers such as EUDRAGIT™ RS and EUDRAGIT™ RL (Rohm Pharma) are suitable for use in the controlled release formulations of the present invention. These polymers are insoluble in pure water, dilute acids, buffer solutions, or digestive fluids over the entire physiological pH range. The polymers swell in water and digestive fluids independently of pH. In the swollen state they are then permeable to water and dissolved actives. The permeability of the polymers depends on the ratio of ethylacrylate (EA), methyl methacrylate (MMA), and trimethylammoriioethyl methacrylate chloride (TAMCI) groups in the polymer. Those polymers having EA:MMA:TAMCI ratios of 1:2:0.2 (EUDRAGIT™ RL) are more permeable than those with ratios of 1:2:0.1 (EUDRAGIT™ RS). Polymers of EUDRAGIT™ RL are insoluble polymers of high permeability. Polymers of EUDRAGIT™ RS are insoluble films of low permeability.

The ammonio methacrylate co-polymers may be combined in any desired ratio. For example, a ratio of EUDRAGIT™ RS: EUDRAGIT™ RL (90:10) may be used. The ratios may furthermore be adjusted to provide a delay in release of the drug. For example, the ratio of EUDRAGIT™ RS: EUDRAGIT™ RL may be about 100:0 to about 80:20, about 100:0 to about 90:10, or any ratio in between. In such formulations, the less permeable polymer EUDRAGIT™ RS would generally comprise the majority of the polymeric material.

The ammonio methacrylate co-polymers may be combined with the methacrylic acid co-polymers within the polymeric material in order to achieve the desired delay in release of the drug. Ratios of ammonio methacrylate co-polymer (e.g., EUDRAGIT™ RS) to methacrylic acid co-polymer in the range of about 99:1 to about 20:80 may be used. The two types of polymers can also be combined into the same polymeric material, or provided as separate coats that are applied to a core.

In addition to the EUDRAGIT™ polymers described above, a number of other such copolymers may be used to control drug release. These include methacrylate ester co-polymers (e.g., EUDRAGIT™ NE 30D). Further information an the EUDRAGIT™ polymers can be found in “Chemistry and Application Properties of Polymethacrylate Coating Systems,” in Aqueous Polymeric Coatings for Pharmaceutical Dosage Forms, ed, James McGinity, Marcel Dekker Inc., New York, pg 109-114).

The coating membrane may further comprise one or more soluble excipients so as to increase the permeability of the polymeric material. Suitably, the soluble excipient is selected from among a soluble polymer, a surfactant, an alkali metal salt, an organic acid, a sugar, and a sugar alcohol. Such soluble excipients include, but are not limited to, polyvinyl pyrrolidone, polyethylene glycol, sodium chloride, surfactants such as sodium lauryl sulfate and polysorbates, organic acids such as acetic acid, adipic acid, citric acid, fumaric acid, glutaric acid, malic acid, succinic acid, and tartaric acid, sugars such as dextrose, fructose, glucose, lactose and sucrose, sugar alcohols such as lactitol, maltitol, mannitol, sorbitol and xylitol, xanthan gum, dextrins, and maltodextrins. In some embodiments, polyvinyl pyrrolidone, mannitol, and/or polyethylene glycol can be used as soluble excipients. The soluble excipient(s) may be used in an amount of from about 1% to about 10% by weight, based on the total dry weight of the polymer.

In one embodiment the slow-release composition comprises aspirin (preferably as a seed of size 0.1 to 1 mm) coated with about 60 parts of a copolymer of ethyl acrylate and methyl acrylate (eg 70:30 ethyl acrylate:methyl acrylate such as EUDRAGIT™ NE30D of high molecular weight (eg about 800,000) and 10 to 20 parts hydroxypropylmethyl cellulose and optionally fillers.

The statin may be incorporated in a coating but is preferably formed as a separate granule combined into a single dosage form with the aspirin component.

In one embodiment, the polymeric material comprises one or more water-insoluble polymers, which are also insoluble in gastrointestinal fluids, and one or more water-soluble pore-forming compounds. For example, the water-insoluble polymer may comprise a terpolymer of polyvinyl polyvinylacetate, and/or polyvinylalcohol. Suitable water-soluble pore-forming compounds include, but are not limited to, saccharose, sodium chloride, potassium chloride, polyvinylpyrrolidone, and/or polyethyleneglycol. The pore-forming compounds may be uniformly or randomly distributed throughout the water insoluble polymer. Typically, the pore-forming compounds comprise about 1 part to about 35 parts for each about 1 to about 10 parts of the water insoluble polymers.

When such dosage forms come into contact with the dissolution media (e.g., intestinal fluids), the pore-forming compounds within the polymeric material dissolve to produce a porous structure through which the drug can diffuse. Such formulations are described in more detail in U.S. Pat. No. 4,557,925, which is herein incorporated by reference for this purpose. The porous membrane may also be coated with a functional coating, as described herein, to inhibit release in the stomach.

In one embodiment, such pore forming controlled release dosage forms comprise simvastatin and/or lovastatin; a filler, such as starch, lactose, or microcrystalline cellulose (AVICEL™); a binder/controlled release polymer, such as hydroxypropyl methylcellulose or polyvinyl pyrrolidone; a disintegrant, such as, EXPLOTAB™, crospovidone, or starch; a lubricant, such as magnesium stearate or stearic acid; a surfactant, such as sodium lauryl sulphate or polysorbates; and a glidant, such as colloidal silicon dioxide (AEROSIL™) or talc.

The polymeric material may also include one or more auxiliary agents such as fillers, plasticizers, and/or anti-foaming agents. Representative fillers include talc, fumed silica, glyceryl monostearate, magnesium stearate, calcium stearate, kaolin, colloidal silica, gypsum, micronized silica, and magnesium trisilicate. The quantity of filler used typically ranges from about 2% to about 300% by weight, and can range from about 20 to about 100%, based on the total dry weight of the polymer. In one embodiment, talc is the filler. In one embodiment, the anti-foaming agent is simethicone. The amount of anti-foaming agent used typically comprises from about 0% to about 0.5% of the final formulation.

The coating membranes, and functional coatings as well, can also include a material that improves the processing of the polymers. Such materials are generally referred to as plasticizers and include, for example, adipates, azelates, benzoates, citrates, isoebucates, phthalates, sebacates, stearates and glycols. Representative plasticizers include acetylated monoglycerides, butyl phthalyl butyl glycolate, dibutyl tartrate, diethyl phthalate, dimethyl phthalate, ethyl phthalyl ethyl glycolate, glycerin, ethylene glycol, propylene glycol, triacetin citrate, triacetin, tripropinoin, diacetin, dibutyl phthalate, acetyl monoglyceride, polyethylene glycols, castor oil, triethyl citrate, polyhydric alcohols, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidized tallate, triisoctyl trimellitate, diethylhexyl phthalate, di-n-octyl phthalate, di-1-octyl phthalate, di-1-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, glyceryl monocaprylate, and glyceryl monocaprate. In one embodiment, the plasticizer is dibutyl sebacate. The amount of plasticizer used in the polymeric material typically ranges from about 10% to about 50%, for example, about 10, 20, 30, 40 or 50%, based on the weight of the dry polymer.

The amount of polymer to be used in the membrane-controlled formulations is typically adjusted to achieve the desired drug delivery properties, including the amount of drug to be delivered, the rate and location of drug delivery, the time delay of drug release, and the size of the multiparticulates in the formulation. The amount of polymer applied typically provides an about 10 to about 100% weight gain to the cores. In one embodiment, the weight gain from the polymeric material ranges from about 25 to about 70%.

The combination of all solid components of the polymeric material, including co-polymers, fillers, plasticizers, and optional excipients and processing aids, typically provides an about 10 to about 450% weight gain on the cores. In one embodiment, the weight gain is about 30 to about 160%.

The polymeric material can be applied by any known method, for example, by spraying using a fluidized bed coater (e.g., Wurster coating) or pan coating system. Coated cores are typically dried or cured after application of the polymeric material. Curing means that the multiparticulates are held at a controlled temperature for a time sufficient to provide stable release rates. Curing can be performed, for example, in an oven or in a fluid bed drier. Curing can be carried out at any temperature above room temperature.

A sealant or barrier can also be applied to the polymeric coatings, including both the functional coatings and the membrane coatings. A sealant or barrier layer may also be applied to the core prior to applying the polymeric membrane coating material. A sealant or barrier layer is not intended to modify the release of simvastatin and/or lovastatin. Suitable sealants or barriers are permeable or soluble agents such as hydroxypropyl methylcellulose, hydroxypropyl cellulose, hydroxypropyl ethylcellulose, and xanthan gum.

Other agents can be added to improve the processability of the sealer or barrier layer. Such agents include talc, colloidal silica, polyvinyl alcohol, titanium dioxide, micronized silica, fumed silica, glycerol monostearate, magnesium trisilicate and magnesium stearate, or a mixture thereof. The sealant or barrier layer can be applied from solution (e.g., aqueous) or suspension using any known means, such as a fluidized bed coater (e.g., Wurster coating) or pan coating system. Suitable sealants or barriers include, for example, OPADRY WHITE Y-1-7000 and OPADRY OY/B/28920 WHITE, each of which is available from Colorcon Limited, England.

The present invention also provides an oral dosage form comprising a formulation as hereinabove defined, in the form of caplets, capsules, particles for suspension prior to dosing, sachets, or tablets. The dosage form can be of any shape suitable for oral administration of a drug, such as spheroidal, cube-shaped oval, or ellipsoidal. The dosage forms can be prepared from the multiparticulates in a manner known in the art and include additional pharmaceutically acceptable excipients, as desired.

In one embodiment of the invention the composition is formed from granules and comprises at least two granule formulations of at least one of aspirin and statin including a first slow-release formulation and a second slow-release formulation a majority of which is time to be released after release of at least a majority of the first formulation. For example the composition may comprise a first aspirin slow-release formulation at least 60% of which is to be released within 8 hours and a second opinion formulation at least 60% of which is to be released in more than eight hours. The aspirin, statin or the aspirin and statin may be formulated in this way.

The composition of the invention may be formulation to provide no more than 80% release of each of aspirin and statin over a period of at least six and more preferably at least eight hours from a single unit dosage.

The unit dosage composition of the invention may provide a physiologically effective release rate over a period of at least six, more preferably at least 8, still more preferably at least 12 and most preferably at least 16 hours.

A single unit dosage may provide the total daily dosage of the combined aspirin and statin or may be administered twice daily. Most preferably the unit dosage is administered once daily.

The invention will now be described with reference to the following examples. It is to be understood that the examples are provided by way of illustration of the invention and that they are in no way limiting to the scope of the invention.

### EXAMPLES

### Example 1

This example relates to the formation of a slow-release tablet containing low doses of simvastatin and aspirin.

Unit dose forms of the composition of the invention may be prepared by forming tablets or filling capsules using batches of controlled-release simvastatin and aspirin granules of the following composition.

| Aspirin Granules | | | Simvastatin Granules | | |
|---|---|---|---|---|---|
| Ingredient | Amount | | Ingredient | Amount | |
| Aspirin (80 mesh) | 80 | g | Simvastatin | 5 | g |
| Hydrogenated Cotton | 30 | g | Lactose | 45.6 | g |
| Seed oil | | | | | |
| Lactose | 15 | g | “Avicel” PH101 | 22.7 | g |
| Hydroxy propyl cellulose | 3 | g | Sulfate | 1 | g |
| (“Methocel” E50) | | | Sodium lauryl sulfate | | |
| Cellulose acetate | 1 | g | “Methocel” KIIOLV | 20 | g |
| phthalate | | | | | |
| Denatured ethanol | 40 | ml | Colloidal silicon dioxide | 0.2 | g |
| Methylene chloride | 40 | ml | Magnesium stearate | 0.5 | g |
| Talc | 3 | g | PVP | 5 | g |
| | | | Isopropyl alcohol | 20 | ml |

Granules containing aspirin and simvastatin may be separately prepared in a planetary Hobart mixer.

Aspirin cotton seed oil and lactose may be deaggregated and placed in a Hobart mixer. The hydroxypropyl cellulose and cellulose acetate phthalate are added to the solvents with mixing and the polymer mixture then added slowly until a wet granular mix is formed. The mixture is dried and screened to provide granules of size of about 100 to 500 microns.

The simvastatin granules may be prepared by dissolving simvastatin in PVP and adding the PVP solution to a mixture of the remaining components to provide a granular wet mix which is dried and screened to provide granules of 100 to 500 microns.

The dried granules of simvastatin and aspirin are mixed and compressed to form tablets containing 80 mg of aspirin and 5 mg of simvastatin.

Alternatively granules may be filled into gelatine capsules to provide an equivalent dosage.

### Example 2

The aspirin granules of Example 1 may be replaced with coated aspirin seeds formed as described in Example 1 of U.S. Pat. No. 4,970,081 using HPMC EUDRIGIT™ NE 30D polymer coatings. The resulting granules may be formed into tablets in accordance with Example 2 of U.S. Pat. No. 4,970,081 except that simvastatin granules according to the above Example 1 are added to provide a unit dose form comprising 50 mg of aspirin and 5 mg of simvastatin.

### REFERENCES

Brantmark B, Wahlin-Boll E, Melander A.
(Bioavalability of acetyl salicylic acid and salicylic acid from rapid and slow release formulations and in combination with dipyridamole; Eur. J. Clin. Pharmacol. 1982; 22(4): 309-14)
Carlsson I, Benthin G, Petersson A S, Wennmalm A.
Differential inhibition of thromboxane A2 and prostacyclin synthesis by low-dose acetylsalicylic acid in atherosclerotic patients.
Thromb Res. 1990 Feb. 1; 57(3):437-44.
James M J, Walsh J A, Foreman R K.
Effect of 50 mg enteric-coated aspirin (Astrix) on thromboxane and prostacyclin synthesis.
Aust N Z J. Surg. 1987 October; 57(10):763-6.
Kyrle P A, Minar E, Brenner B, Eichler H G, Heistinger M, Marosi L, Lechner K.
Thromboxane A2 and prostacyclin generation in the microvasculature of patients with atherosclerosis—effect of low-dose aspirin.
Thromb Haemost. 1989 Jun. 30; 61(3):374-7.
Roberts M S, Joyce R M, McLeod L J, Vial J H, Seville P R
Slow-release aspirin and prostaglandin inhibition.
Lancet 1986 May 17: 1(8940): 1153-4
Stepien J M, Prideaux R M, Willoughby S R, Chirkov Y Y, Horowitz J D.
Pilot study examining the effect of cholesterol lowering on platelet nitric oxide responsiveness and arterial stiffness in subjects with isolated mild hypercholesterolaemia.
Clin Exp Pharmacol Physiol. 2003 July; 30(7):507-12.
Smith H J
Liver-selective Therapy
1999
US Patent Application 20020160044

1. An oral composition for treatment or prevention of atherosclerosis comprising a low-dose aspirin and a low-dose of statin wherein the aspirin and statin are in a slow-release formulation.

2. An oral composition according to claim 1 wherein the statin is selected from poorly water-soluble statins.

3. An oral composition according to claim 2 wherein the statin is selected from simvastatin and lovastatin.

4. An oral composition according to claim 1 wherein the dose and delivery rate according to claim 1 of each of aspirin and statin are sufficient to achieve a clinical effective amount in the portal vein and insufficient following hepatic clearance to produce systemic effect.

5. An oral composition according to claim 1 wherein the composition comprises a unit dosage form containing aspirin in an amount in the range of from 5 to 100 mg a statin in an amount in the range of from 0.5 to 100 mg per day.

6. An oral dosage composition according to claim 5 wherein the unit dosage form comprises aspirin in an amount in the range of from 5 to 50 mg and a statin in an amount in the range of from 5 to 50 mg.

7. An oral dosage composition according to claim 6 wherein the unit dosage form is selected from tablets and capsules.

8. An oral composition according to claim 1 wherein the statin and aspirin are separately formulated as granules and combined to provide a unit dosage in the form of a capsule or tablet.

9. An oral composition according to claim 1 where the release of both aspirin and statin is spread over more than 8 hours and preferably more than 12 hours.

10. An oral composition according to claim 1 wherein no more than 50% of each of statin and aspirin is released within the first 6 hours.

11. An oral composition according to claim 1 comprising a slow-release matrix or coating comprising a water soluble polymer and/or water insoluble polymer.

12. An oral composition according to claim 1 wherein the composition comprises slow-release granules of statin and slow-release granules of aspirin compressed to form a tablet or providing a filling for capsules.

13. A method for treatment or prevention of atherosclerosis comprising orally administering to a patient
(a) statin in an amount and at a release rate sufficient to provide a level in the portal vein sufficient to reduce cholesterol and wherein the amount following hepatic clearance is insufficient to provide a clinically effect in the systemic vasculature to thereby provide a selective cholesterol lowering effect in the liver; and
(b) aspirin in an amount sufficient to provide a level in the portal vein to decrease platelet adhesiveness and wherein the amount is sufficiently low following hepatic clearance to avoid significantly suppressing production of prostacyclin to provide a platelet selective effect.

14. A method according to claim 13 wherein the statin is a low water solubility statin.

15. A method according to claim 13 wherein the statin is selected from simvastatin and lovastatin.

16. A method according to claim 13 wherein the unit dosage form is administered comprising both the statin and aspirin.

17. A method according to claim 16 wherein the unit dosage form is selected from tablets and capsules comprising slow-release granules of each of statin and aspirin.

18. A method according to claim 13 wherein no more than 50% of each of the statin and aspirin is released after 2 hours.

19. A method according to claim 18 wherein no more than 50% of each of the statins and aspirin is released after 3 hours.

20. A method according to claim 13 wherein the statin is administered in an amount of from 5 to 80 mg per day and the aspirin is administered in an amount of from 10 to 100 mg per day.

21. A method according to claim 19 wherein each of the statin and aspirin are administered in an amount of from 10 to 80 mg per day.

22. A method according to claim 13 wherein the statin and aspirin are formulated with a slow-release polymer.

23. A method according to claim 11 wherein less than 5% of each of the statins and aspirin are systemically bio available following hepatic clearance.
